(19) United States
(12) Patent Application Publication
(10) Pub. No.: US 2008/0021312 A1
(43) Pub. Date: Jan. 24, 2008

(54) Needle targeting device

(76) Inventors: Robert M. Olson, Parrish, FL (US); Preston Robert Olson, North Saint Paul, MN (US); John L. Stresing, Delray Beach, FL (US)

Correspondence Address: John F. Klos;FULBRIGHT & JAWORSKI L.L.P., 2100 IDS Center, 80 South Eighth Street, Minneapolis, MN, 55402-2112, US

(21) Appl. No.: 11/489,401

(22) Filed: Jul. 19, 2006

(51) Int. Cl. A61B 5/05

(52) U.S. Cl. 600/429

(57) ABSTRACT

The present invention is directed to a handheld needle targeting device which medical personnel can use to position a needle or K-wire under x-ray guidance, as part of a medical or surgical procedure. The device of this invention can also be used to both hold and advance a needle. The device is at least partially radiopaque, so that at least part of the device is visible under x-ray.

### FIELD OF THE INVENTION

This invention relates to a device used to position a needle in the course of a medical or surgical procedure. More specifically, the invention relates to a handheld device used in the positioning of a needle or Kirschner wire under x-ray guidance.

### BACKGROUND

X-ray, or fluoroscopic, guidance is a useful technique in various medical and surgical procedures involving the insertion of a needle or Kirschner wire (“K-wire”) into a patient. When this technique is used, medical personnel use x-ray images of the patient to determine the correct location on a patient's anatomy to insert the needle or K-wire. For example, as discussed in U.S. Pat. No. 6,726,691 to Osorio, et al., x-ray guidance can be used in vertebroplasty procedures, in which a cavity-forming device is inserted through a needle into a vertebral body. In such procedures, medical personnel can use fluoroscopic x-ray to position the needle so that it is properly inserted into the correct vertebral body. Similarly, x-ray guidance may also be used in surgical procedures to show medical personnel where a K-wire should be inserted into a patient. For example, U.S. Pat. No. 6,827,741 to Reeder discusses the use of fluoroscopy to verify the positioning of a K-wire in the intramedullary canal of the radius.

In the past, procedures using x-ray guidance have involved taking an x-ray, positioning a needle, taking another x-ray, moving the needle if it is in an incorrect position, and repeating this process until the needle is positioned at the correct location. This process can involve exposing a patient to x-rays for an undesirably long period of time. If medical personnel were to guide the needle into position by hand under x-ray guidance, it could reduce the amount of time that the patient was exposed to x-rays, but would subject the medical personnel to x-rays themselves.

Instrumentation has been developed to improve the efficiency of the use of x-rays in needle positioning. For example, U.S. Pat. No. 4,875,478 to Chen describes the use of a compression grid and needle holder, with an x-ray source, to accurately insert a biopsy needle into a breast lesion. Automated needle targeting systems have also been developed. However, such instrumentation is expensive, and is not always easily adapted to a variety of applications.

### SUMMARY OF INVENTION

The present invention is directed to a handheld needle targeting device which medical personnel can use to position a needle or K-wire under x-ray guidance. The device is at least partially radiopaque, so that at least part of the device is visible under x-ray.

In one embodiment, the subject invention includes two arms which are connected at the curved proximal end. A living hinge, located between the proximal and distal ends of the device, extends between the two arms. The arms extend to the distal end of the device, which is rounded to form a circular shape. The distal end includes a circular aperture, which is adapted to hold a needle or a K-wire. When the arms are squeezed toward each other by applying pressure to them at a point between the proximal end and the living hinge, the living hinge bends. This action causes the two arms to move apart from each other at the distal end, thereby allowing the device to be removed from the needle when the desired needle depth is reached.

The device of this invention allows medical personnel to position and insert a needle under x-ray guidance, while enabling them to reduce x-ray exposure to themselves and to their patients. The device can be used in a variety of medical and surgical applications.

### BRIEF DESCRIPTION OF THE DRAWINGS

For the purposes of facilitating the understanding of the subject matter sought to be protected, there is illustrated in the accompanying drawings an embodiment thereof. From an inspection of the drawings, when considered in connection with the following description, the subject matter sought to be protected, its construction and operation, and many of its advantages should be readily understood and appreciated.

FIG. 1 is a view of an embodiment of a needle targeting device according to the present invention.

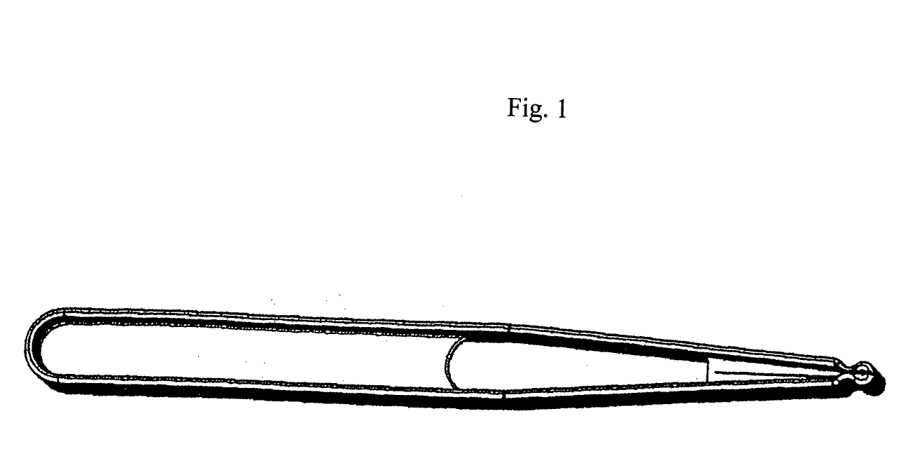

FIG. 2 is a diagrammatic top view of the needle targeting device of FIG. 1.

FIG. 3 is a diagrammatic top view of an embodiment of a needle targeting device, when the device is in the open position.

FIG. 4 is a diagrammatic top view of the distal end of the needle targeting device of FIG. 1.

FIG. 5 is a diagrammatic side view of the needle targeting device of FIG. 1.

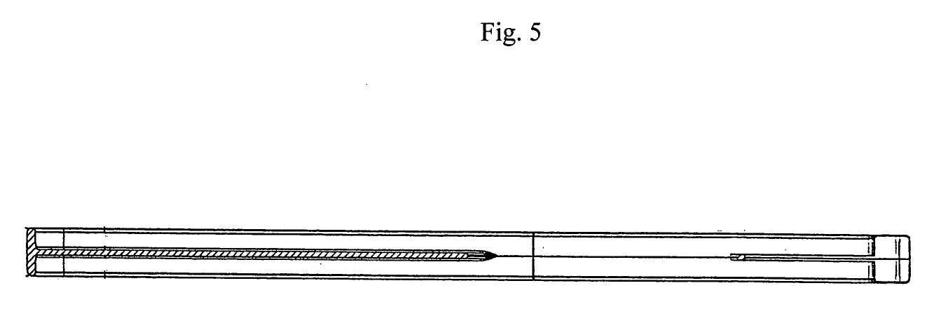

### DETAILED DESCRIPTION

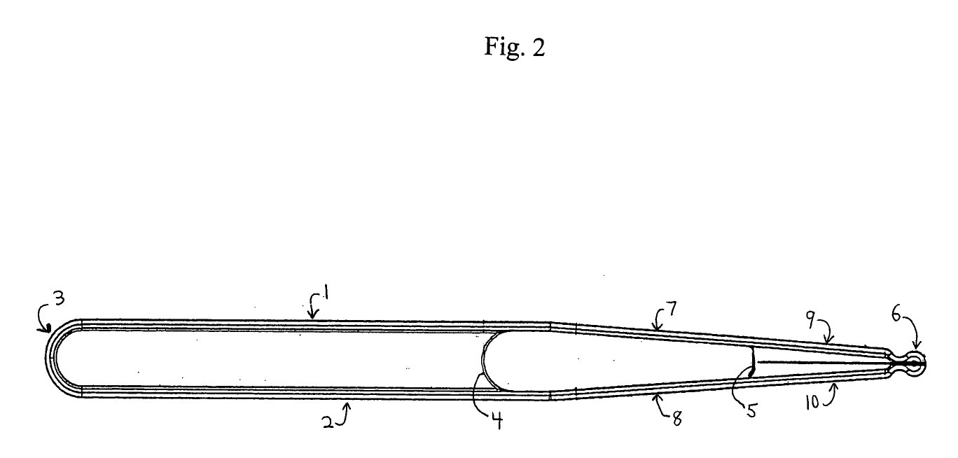

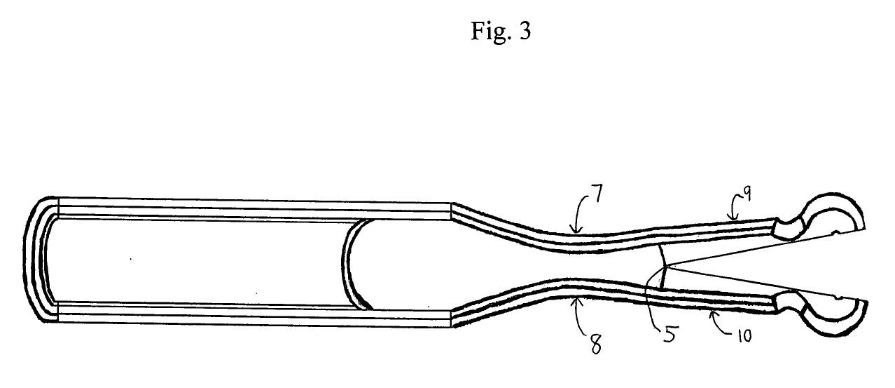

The present invention is directed to a handheld needle targeting device that medical personnel can use to position a needle under x-ray guidance without being exposed to radiation themselves. As shown in FIG. 2, in one embodiment, the needle targeting device includes two arms 1 and 2, which are connected by the curved proximal end 3 of the device, by a connector 4, and by a living hinge 5. The arms extend to the distal end 6 of the device, which is rounded to form a circular shape. When the arms are squeezed toward each other by applying pressure to the arms at sections 7 and 8, the living hinge 5 bends. The action of the living hinge 5 causes sections 9 and 10 of the device, at the distal end, to move apart from each other, as shown in FIG. 3.

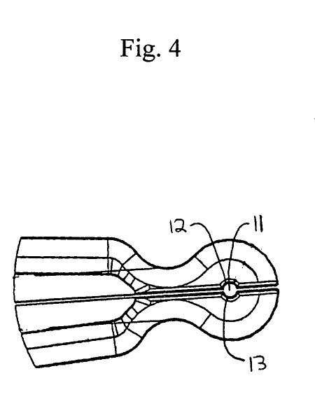

As shown in FIG. 4, at the distal end of one embodiment of the device, the device contains a circular aperture 11. This circular aperture is adapted to hold a needle or K-wire when the device is in the rest position (i.e. when pressure is not being applied to the arms at sections 7 and 8). A resilient material, which facilitates the gripping of the needle or K-wire, is located along the edges 12 and 13 of the circular aperture.

In one embodiment, the needle targeting device is approximately 12 inches long, and is comprised of a medical grade plastic compound. In this embodiment, the device has a metal flake exterior around at least part of the aperture at the distal end. Alternatively, the device can be impregnated with, or loaded with, radiopaque materials, such as bismuth or stainless steel, around at least part of the circular aperture of the device. Therefore, the device is radiopaque at the portion of the device where the needle is held, allowing that portion of the device to be viewed under x-ray.

The device of this invention can be made out of materials which ensure that the device is cost effective. The device can also be made out of materials that allow the device to be disposable after one use.

The aperture, and the resilient material attached to the edges of the aperture, may be designed such that a single device is able to hold needles of various sizes. For example, a single device could be used to hold either 19 gauge, 22 gauge, or 25 gauge needles.

Although the following discussion refers to the use of the targeting device with a needle, the device can also be used with a K-wire in the same manner.

When the needle targeting device is used to localize the starting point of a needle, the device can be placed on the patient's skin, or above the surface of the patient's skin. Holding the device in the hand that will not hold the needle, the device is moved under fluoroscopic guidance until the distal end is positioned at the point on the patient's anatomy where the needle is to be inserted. The device is then oriented such that the area around the circular aperture forms a round circle over the desired part of the anatomy. This circle will show the location where the needle is to be inserted.

Next, the needle is placed in the aperture at the distal end of the targeting device. The needle is then advanced into the patient. As the needle is advanced, its position can be monitored using live, pulsed, or spot fluoroscopy. The position of the needle can be adjusted if this monitoring indicates that adjustment is required. When the desired needle depth is reached, the targeting device can be disengaged from the needle by squeezing the device at a point on the arms between the living hinge and the proximal end, thereby causing the arms to move apart from each other at the distal end, such that the device is no longer holding the needle.

The needle targeting device of this invention may be used in a variety of different medical and surgical procedures. Examples of procedures in which the needle targeting device may be used to position needles or K-wires are as follows:

Cervical, thoracic and lumbar epidural steroid injections;

Selective nerve root blocks;

Transforaminal cervical and lumbar blocks;

Thoracic blocks;

Cervical, lumbar and thoracic facet injections;

Radio frequency rhizotomy;

Cryoablation procedures;

Intradiscal Electrothermal Annuloplasty (“IDET”) procedures;

Discography;

Intrathecal catheter placement;

Spinal cord stimulators;

Kyphoplasty;

Vertebroplasty;

Nucleoplasty;

Trigger point injections;

Sacro-iliac injections;

Arthrography;

Epiduragrams;

Myelography;

Stellate ganglion blocks;

Sympathetic blocks;

Costovertebral joint blocks;

Neurolytic procedures;

Joint injections;

Celiac plexus blocks;

Electrotherapy including Transcutaneous Electrical Nerve Stimulation (“TENS”);

Percutaneous laser disc decompression;

Medial branch blocks;

Any pain management procedure in which fluoroscopy is used;

Orthopedic surgery;

Surgery in which a K-wire is used; and,

Foreign body removal.

The needle targeting device of this invention may also be used in minimally invasive spine surgery. It can be used in any procedure in which a needle or K-wire is used in the beginning of the procedure, in order to localize the correct level of anatomy. Specific examples of spine surgery procedures which may use the needle targeting device of this invention include: Posterior Lumbar Interbody Fusion (“PLIF”), Anterior Lumbar Interbody Fusion (“ALIF”), percutaneous disc decompression, and pedicle screw placement.

The examples provided in this specification are not intended to be an exhaustive list of procedures in which the needle targeting device of this invention may be used. Those of skill in the art may find uses for the needle targeting device which are not mentioned in this specification.

The needle targeting device of this invention allows medical personnel to plan the starting point of needle or K-wire insertion. This device can also be used to both hold and advance a needle, unlike a hemostat or clamp.

The device of this invention allows medical personnel to position and insert a needle under fluoroscopic guidance, while enabling them to reduce x-ray exposure to themselves. By holding the device near its proximal end, they are able to keep their hands away from the direct x-ray beam while the device is being positioned, and while the needle is being inserted into the patient. The device also allows medical personnel to reduce their patients' exposure to x-rays, because the overall amount of fluoroscopic time associated with the positioning and insertion of the needle is reduced. The time is reduced because the device allows medical personnel to plan the trajectory of the needle, thereby facilitating proper needle placement in a reduced amount of time.

Although the present invention and it advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, the processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A needle targeting device, comprising:
a first arm and a second arm, wherein a living hinge is connected to the first arm and the second arm at a location between a proximal end and a distal end of the device; wherein the distal end comprises an aperture adapted to hold a needle; wherein the device is adapted such that squeezing the first arm and the second arm toward each other, at a location between the living hinge and the proximal end, causes the first arm and the second arm to separate between the living hinge and the distal end.

2. The needle targeting device of claim 1, wherein the living hinge extends between the first arm and the second arm.

3. The needle targeting device of claim 1, wherein the device is comprised of a resilient plastic material.

4. The needle targeting device of claim 1, wherein the device comprises a radiopaque material around at least part of the aperture.

5. The needle targeting device of claim 4, wherein the radiopaque material substantially forms a circle.

6. The needle targeting device of claim 1, wherein the device is coated with a radiopaque material around at least part of the aperture.

7. The needle targeting device of claim 6, wherein the radiopaque material substantially forms a circle.

8. The needle targeting device of claim 1, wherein a resilient material is attached to an edge of the aperture, such that the resilient material is capable of gripping a needle.

9. A needle targeting device, comprising:
a first arm and a second arm, wherein a living hinge is connected to the first arm and the second arm at a location between a proximal end and a distal end of the device; wherein the distal end comprises an aperture adapted to hold a wire; wherein the device is adapted such that squeezing the first arm and the second arm toward each other, at a location between the living hinge and the proximal end, causes the first arm and the second arm to separate between the living hinge and the distal end.

10. The needle targeting device of claim 9, wherein the living hinge extends between the first arm and the second arm.

11. The needle targeting device of claim 9, wherein the device is comprised of a resilient plastic material.

12. The needle targeting device of claim 9, wherein the device comprises a radiopaque material around at least part of the aperture.

13. The needle targeting device of claim 12, wherein the radiopaque material substantially forms a circle.

14. The needle targeting device of claim 9, wherein the device is coated with a radiopaque material around at least part of the aperture.

15. The needle targeting device of claim 14, wherein the radiopaque material substantially forms a circle.

16. The needle targeting device of claim 9, wherein a resilient material is attached to an edge of the aperture, such that the resilient material is capable of gripping a needle.

17. A method of using a needle targeting device, comprising:
moving the device under fluoroscopic guidance until a distal end of the device is positioned at a location on a patient where a needle is to be inserted;
orienting the device such that an edge of an aperture of the distal end of the device forms a circle over the location on the patient where the needle is to be inserted;
placing the needle into the aperture of the device; and,
advancing the needle into the patient.

18. The method of claim 17, further comprising:
monitoring a position of the needle using fluoroscopy, as the needle is advanced into the patient.

19. The method of claim 18, further comprising:
adjusting the position of the needle when monitoring indicates that adjustment is required.

20. The method of claim 17, further comprising:
disengaging the device from the needle by squeezing two arms of the device toward each other, at a point between a living hinge and a proximal end of the device, thereby causing the two arms to move apart from each other at the distal end of the device, such that the device is no longer holding the needle.
